(19) United States
(12) Patent Application Publication
(10) Pub. No.: US 2011/0308521 A1
(43) Pub. Date: Dec. 22, 2011

(54) VAPORIZER

(76) Inventor: Mark Kofford, Dana Point, CA (US)

(21) Appl. No.: 12/820,695

(22) Filed: Jun. 22, 2010

(51) Int. Cl. A61M 16/10

(52) U.S. Cl. 128/203.27

(57) ABSTRACT

Provided is a vaporizing device configured for use with a disposable bottle to employ the disposable bottle as a collection device for collecting and delivering the vapor to a user. The vaporizing device includes a threaded connector portion configured to engage with the threaded bottle connector found on a conventional beverage container, such as a water bottle, soda bottle, sports drink bottle, or the like. The vaporizing device is configured to receive and heat herbal contents to produce a vapor for inhalation by a user.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates generally to an herbal vaporization device, and more specifically to a thermal vaporization device sized and configured to engage with a conventional beverage bottle for collecting and delivering the herbal vapors to a user.

Cigarettes or smoking pipes are commonly used for inhaling cannabinoids, nicotine, and other active ingredients of commonly smoked herbal materials. Although smoking is an effective means of delivering the active ingredients to the user, it is recognized that smoking may additionally deliver numerous irritants and possible carcinogens along with the desired active ingredients to the user, especially within the tracheobronchial tree and oral cavity.

Given the dangers associated with smoking herbal materials, there is a desire for alternate means of receiving the desirable ingredients of the herbal contents, while mitigating delivery of the irritants and possible carcinogens. It is believed that vaporization of the herbal materials is effective toward achieving that end. In particular, vaporization, the temperature range of which is related to the boiling points of the desired active constituents, is believed to separate the desirable, active constituents, from the non-desirable and harmful constituents. Vaporization of herbal products is typically achieved by heating the herbal products to a temperature wherein the desirable constituents are contained within the vapor, which is then captured and delivered to the user.

Common vaporization devices typically include a bowl configured to receive and heat the herbal contents to produce the vapor. The bowl may be heated by a flame, such as by a lighter or match. Once the herbal contents vaporize, the vapor is collected within a collection device for delivery to the user. The collection device is in fluid communication with the bowl to receive the vapor once it is generated. The collection device typically includes a mouthpiece for enabling a user to inhale the collected vapor.

When the vapor is collected within the collection device, the vapor may produce a film on the inner surface of the collection device, which may pollute or contaminate the collection device. Although the collection device may be cleaned, the film may form after only a single use. Accordingly, the collection device may require cleaning after each use, which may become tedious.

Therefore, in view of the foregoing, there is a need in the art for a vaporization device having a disposable collection device. The present invention addresses these particular needs, as will be discussed in more detail below.

### BRIEF SUMMARY

There is provided a vaporizing device configured for use with a disposable bottle to employ the disposable bottle as a collection device for collecting and delivering the vapor to a user. The vaporizing device includes a threaded connector portion configured to engage with the threaded bottle connector found on a conventional beverage container, such as a water bottle, soda bottle, sports drink bottle, or the like.

More specifically, the vaporizer device includes a housing having a connector portion defining threads configured to be engageable with the threaded bottle connector to connect the bottle to the housing. An herbal receptacle is connected to the housing and includes a base and a wall extending from the base to define a receptacle cavity for receiving the herbal products. A heating member is in thermal communication with the herbal receptacle and is configured to heat the herbal receptacle for vaporizing the herbal products therein.

The housing may be rotatable relative to the bottle between an engaged configuration and a disengaged configuration. The threads of the housing may be engaged with the threaded bottle connector when in the engaged configuration. Furthermore, the threads of the housing may be disengaged from the threaded bottle connector when in the disengaged configuration.

The heating member may include an elongate heating rod. The vaporizer device may further include a thermal conduit connected to the herbal receptacle. The thermal conduit may include an inner wall defining a thermal opening with the elongate heating rod being insertable into the thermal opening.

The vaporizer device may comprise a power unit in electrical communication with the heating member for supplying electrical power to the heating member. A temperature regulator may be in electrical communication with the heating member and the power unit. The temperature regulator may be configured to control the flow of electrical power from the power unit to the heating member to maintain the temperature of the herbal receptacle within a vaporizing temperature range. A temperature indicator may be in operative communication with the temperature regulator and configured to emit a temperature signal when the temperature of the receptacle is in the vaporizing temperature range. A power switch may be in operative communication with the power unit, with the power switch being disposable in an ON position and an OFF position. A power indicator may be in operative communication with the power switch to emit a power signal when the power switch is in the ON position.

There may also be provided a method of vaporizing an herbal product comprising the steps of providing a collection bottle and a vaporizing device, as described above. The collection bottle includes a bottle wall defining a collection enclosure and a threaded bottle connector. A cooling aperture and an inhaling aperture are punctured within the bottle wall at opposing end portions of the collection bottle. The bottle is then connected to the vaporizing device by engaging the threaded bottle connector to the connector portion of the housing.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a cross sectional view of a vaporizer device having a bottle engaged therewith;

FIG. 2 is an upper perspective view of the vaporizer device and bottle depicted in FIG. 1;

FIG. 3 is an upper perspective view of the vaporizer device with the bottle being disengaged therefrom; and

FIG. 4 is a top view of the vaporizer device depicted in FIG. 3.

### DETAILED DESCRIPTION

The detailed description set forth below in connection with the appended drawings is intended as a description of certain embodiments of the present disclosure, and is not intended to represent the only forms that may be developed or utilized. The description sets forth the various functions in connection with the illustrated embodiments, but it is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

Referring now to the drawings, there is depicted a vaporizer device 10 configured for use with a bottle 12, such as a conventional water bottle, soda bottle, sports drink bottle, or the like. The vaporizer device 10 is configured to vaporize herbal products 14, such as marijuana or tobacco, and the bottle 12 is connectable to the vaporizer device 10 to collect the vapor 16 for inhalation by a user. It is understood that vaporizing herbal products 14 tends to mitigate the transfer of harmful portions of the herbal products 14 to the user, when compared to traditional smoking of the herbal products 14. For instance, carcinogenic particles in marijuana and tobacco are believed to be transferred to the user when the user burns the marijuana or tobacco for smoking. Conversely, when the marijuana or tobacco is vaporized, the useful portions of those products are transferred to the user in the vapor, while the harmful portions are not as likely to be transferred.

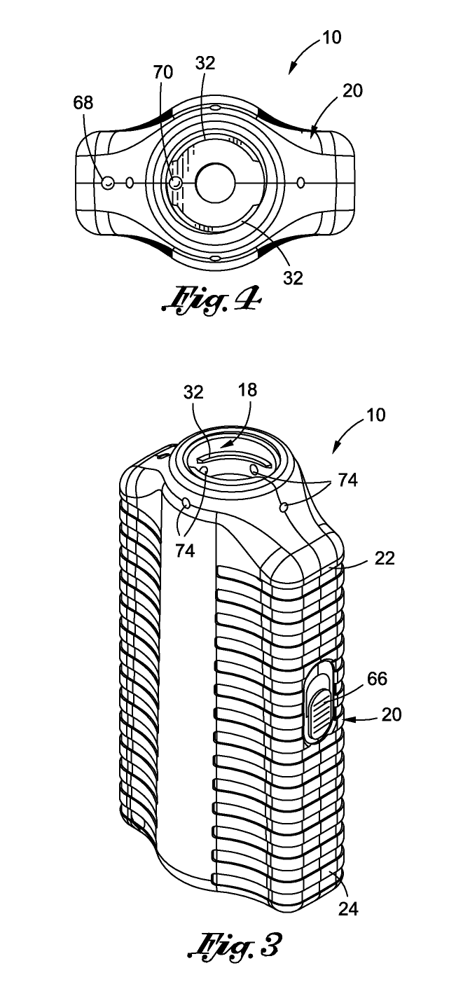

The vaporizer device 10 includes a threaded connector portion 18 (see FIG. 3) which is engageable with the threaded end of the bottle 12. The bottle 12 may be easily attached to the vaporizer device 10 for use, and subsequently easily detached from the vaporizer device 10 after use. It is understood that a film may form on the inner surface of the bottle 12 after an herbal product 14 is vaporized. Therefore, when the bottle 12 becomes dirty, it can quickly and easily be removed and replaced with a new clean bottle 12.

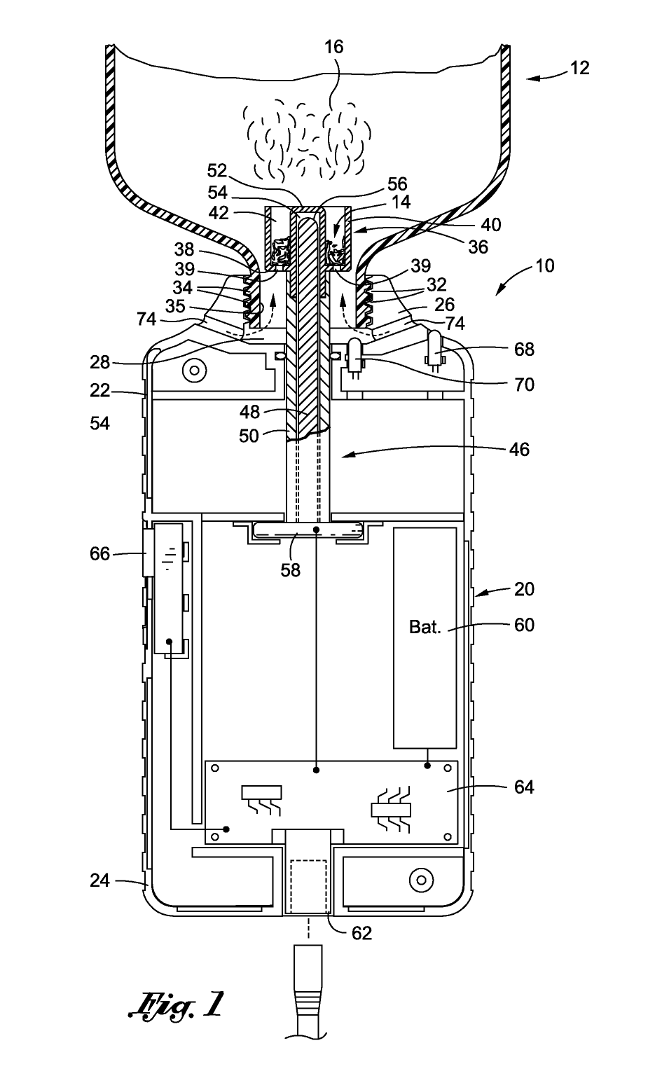

The vaporizer device 10 includes a housing 20 configured to be hand portable and easily grippable by a user. The housing 20 includes an upper end portion 22 (see FIGS. 1 and 3) and an opposing lower end portion 24 (see FIGS. 1 and 3). The housing 20 further includes a cylindrical medial portion and a tapered thickness extending from the medial portion laterally outward. The outer surface of the housing 20 may include a series of ribs or grooves to provide an easily grippable surface for the user. The housing 20 may be formed out of two molded halves which are coupled to each other, such as by snap-fit engagement, or by a mechanical fastener, e.g., screws. The housing 20 may be fabricated out of a lightweight and durable material, such as plastic, or metal.

The housing 20 includes a housing wall 26 (see FIG. 1) defining a housing opening 28 (see FIG. 1) adjacent the upper end portion 22, which extends into a housing cavity. Internal threads 32 are formed within the housing opening 28 to facilitate engagement with the bottle 12. It is understood that most bottles 12 include external threads 34 formed on a bottle connector 35. The threads 32 formed within the housing opening 28 are sized and configured to engage with the external threads 34 formed on the bottle connector 35. The housing threads 32 may be double locking threads configured to engage with bottles 12 having different size threaded connectors 35. The threads 32 shown in FIG. 1 are configured specifically for the bottle 12 depicted therein, whereas the threads 32 shown in FIGS. 3 and 4 are more universal in nature, and may be used with bottles having different thread sizes.

Although the Figures depict a housing 20 configured to engage with an externally threaded bottle 12, it is contemplated that other embodiments of the housing 20 may be configured for engagement with an internally threaded container. For instance, the housing 20 may include a collar (not shown) disposed about the opening 28, wherein the collar includes external threads configured to engage with the internal threads formed on the container. Alternatively, an adapter may be used to convert an internally threaded housing for engagement with an internally threaded container.

Referring now specifically to FIG. 1, there is depicted a cross sectional view of the vaporizer device 10 and bottle 12. The vaporizer device 10 includes an herbal receptacle 36 having a base 38 and a wall 40 extending from the base 38 to define a receptacle cavity 42 for receiving the herbal products 14. In the embodiment depicted in the drawings, the herbal receptacle 36 defines a cylindrically-shaped wall 40. The base 38 may include a plurality of base apertures 39 formed therein to facilitate a desired fluid flow. In one embodiment, the base apertures 39 are 1 mm in diameter.

The herbal receptacle 36 is disposed in thermal communication with a heating assembly including a heating conduit 46 (see FIG. 1) and a heating rod 48 (see FIG. 1). The heating conduit is preferably a copper tube that is sized to fit over the heating rod 48 to transfer heat from the heating rod 48 to the herbal receptacle. In particular, the heating conduit 46 includes an opening 54, while the heating rod 48 defines a distal tip 56 which is advanced into the opening 54 until a flanged end portion 58 is disposed adjacent the heating conduit 46. The herbal receptacle is disposed adjacent an end portion of the heating conduit 46 to receive heat therefrom. As shown in FIG. 1, the herbal receptacle 36 is integrally connected to the heating conduit 46; however, it is understood that in other embodiments, the herbal receptacle 36 may be detachable from the heating conduit 46. For instance, the herbal receptacle 36 and heating conduit 36 may include complimentary threads to enable threaded engagement therebetween. The herbal receptacle 36 is sized to be inserted through the bottle opening and extend at least partially into the bottle 12 during use. Other implementations may include a herbal receptacle 36 which does not extend into the bottle 12, and may be at least partially disposed within the housing 20.

A power supply 60 is also disposed within the housing 20, and is in electrical communication with the heating rod 48. One implementation of the vaporizer device 10 includes a power supply 60 comprised of a rechargeable battery or batteries. A power port 62 may be disposed within the housing 20, and electrically connected to the rechargeable batteries to allow for recharging of the batteries, similar to recharging of a cell phone, or laptop computer. The power supply 60 may also be comprised of disposable batteries, or a power outlet for connecting to an external power source, such as a power outlet.

As power is supplied to the heating rod 48, the heating rod 48 generates heat, which is transferred to the heating conduit 46 and ultimately to the herbal receptacle 36, preferably via conduction between the heating conduit 46 and the herbal receptacle 36. In other words, the herbal receptacle 36 is preferably disposed in contact with the heating conduit 46 to facilitate heat transfer therebetween.

It is understood that the herbal products 14 vaporize at a vaporization temperature. For instance, with regard to marijuana, approximately 20% of the HTC is released at a vaporization temperature below 250° F. At temperatures between 250° F.-350° F., 25%-40% of the HTC is released, along with a minor amount of carbon. Marijuana burns at approximately 380° F. to release approximately 60% HTC.

Certain aspects of the vaporizer device 10 are directed toward maintaining the temperature of the herbal receptacle 36 within a certain vaporization temperature range. For instance, the vaporization range for marijuana may be from 210° F. to 220° F. The vaporization range may be pre-programmed into the device 10 for various herbal products 14, or alternatively, the device 10 may include a user interface, such as a touch-screen, dial, or the like to allow a user to set the vaporization range. Those skilled in the art will appreciate that a few drops of water, alcohol, or glycol improves the vaporization efficiency at lower temperatures.

Along these lines, the vaporizer device 10 may include a temperature control unit 64 in electrical communication with the power supply 60 and the heating rod 48. A temperature sensor may be in electrical communication with the temperature control unit 64 for measuring the temperature of the herbal receptacle 36, the heating conduit 46, or the heating rod 48 to determine whether the temperature of the herbal receptacle 36 is within the desired vaporization range. The temperature control unit 64 receives a temperature input reading from the temperature sensor, and compares the temperature input reading with the allowable vaporization range.

The vaporizer device 10 further includes an ON/OFF switch 66 to control operation of the device 10. When the switch 66 is in the OFF position, power is restricted from being transferred to the heating rod 48. When the switch 66 is in the ON position, power is sent to the heating rod 48 to increase the temperature of the heating rod 48. When the device 10 is powered up, the heating rod 48 receives power from the power supply 60 until the temperature sensor indicates that the temperature of the herbal receptacle 36 is beyond the upper limit of the allowable temperature range, at which time power is restricted to the heating rod 48. Once the temperature of the herbal receptacle 36 falls below the allowable temperature range, power is then directed to the heating rod 48 to increase the temperature thereof. This power cycle repeats as long as the device is in the ON position to maintain the temperature of the herbal receptacle 36 within the vaporizing temperature range.

The vaporizer device 10 may include a power indicator 68 and a temperature indicator 70 to communicate the operational status (ON or OFF) of the device 10, as well as the temperature of the herbal receptacle 36 to the user. The power indicator 68 may include a light (e.g., light emitting diode) disposed on the outer surface of the housing 20 and emit light when the device is in the ON state (and not emit light when in the OFF state). Furthermore, the temperature indicator 70 may include a light (e.g., light emitting diode) disposed within the housing opening 28 to be visible by a user when the user looks into the housing 20. The temperature light 70 may be configured to emit light when the temperature of the herbal receptacle 36 is outside of the vaporization range. Therefore, if the user notices that the power indicator 68 is on, and the temperature indicator 70 is off, then the temperature of the herbal receptacle 36 is in the vaporization range. It is also understood that as an alternative, the temperature light 70 may emit light when the temperature of the herbal receptacle 36 is within the vaporization range.

The temperature light 70 and power light 68 may emit two different colors of light. In a preferred embodiment, the temperature light 70 is blue, while the power light 68 is red, although those skilled in the art will appreciate that other colors may be used without departing from the spirit and scope of the present invention.

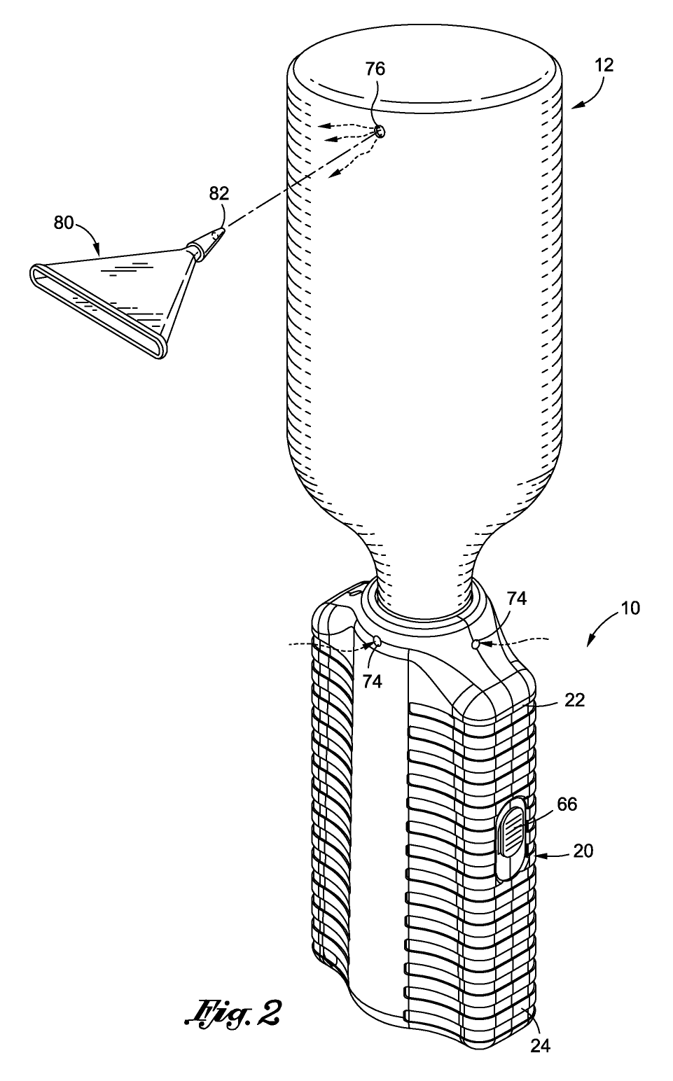

In order to effectuate the desired fluid flow, the housing 20 may include a plurality of ventilation holes 74 (see FIGS. 1-2) formed therein. The ventilation holes 74 may extend through the housing wall 26 to dispose the housing opening 28 in fluid communication with the ambient environment. Therefore, when the user inhales the vapor from the bottle 12, ambient air is drawn into the housing opening 28 through the ventilation holes 74. The ambient air generally cools the vapor before it is inhaled by the user.

Given the elevated temperatures generated by the heating rod 48, one embodiment of the vaporizer device 10 may include an insulative sleeve (not shown) disposed about the body of the heating conduit 46 to protect the adjacent components (e.g., the housing, power unit, etc.) from the elevated temperatures of the heating conduit 46, as well as to mitigate energy loss from the heating conduit 46. The insulative sleeve may be formed out of a ceramic material, or other material configured to mitigate heat transfer from the heating conduit 46.

While the foregoing discussion pertains primarily to the structure of the vaporizer device 10, operation of the vaporizer device 10 is described below. A bottle 12 obtained and is prepared for use with the vaporizer device 10 by puncturing an inhalation hole 76 through the bottle 12 adjacent an opposing end thereof (e.g., the closed end of the bottle 12). A mouthpiece 80 may be used to puncture the inhalation hole 76 in the bottle 12. The mouthpiece 80 may include a pointed end portion 82 for easily piercing through the wall of the bottle 12.

Once the bottle 12 is prepared, herbal products 14 are placed within the herbal receptacle 36, and the bottle 12 is connected to the housing 20 by screwing the bottle connector 35 into the housing opening 28 so the threads on the bottle 12 engage with the threads on the housing 20 to dispose the bottle 12 in fluid communication with the vaporizer device 10. The user then places the ON/OFF switch 66 in the ON position to heat the herbal receptacle 36. Once the herbal receptacle 36 reaches the vaporization temperature, the herbal products 14 vaporizes and accumulates within the enclosure defined by the bottle 12. The user then inhales through the inhalation opening 76, causing ambient air to enter the enclosure to cool the vapor 16. The cooled vapor 16 is then delivered to the user through the inhalation opening 76. When the user is finished, the ON/OFF switch 66 is disposed in the OFF position which causes the heating rod 48 to cool down to an ambient temperature. The ON/OFF switch may be biased into the OFF position to prevent the device 10 from being left in the ON position inadvertently. The temperature control unit 64 may also include an emergency shutoff which powers down the device 10 after a preset period of time. After the device 10 is turned OFF, the bottle 12 is unscrewed from the housing 20, and may be discarded. The device 10 may be plugged in to a power source to recharge the power supply 60 for a subsequent use.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A vaporizer device for vaporizing herbal products for inhalation by a user through a bottle having a threaded bottle connector, the vaporizer device comprising:
a housing having a connector portion defining threads configured to be engageable with the threaded bottle connector to connect the bottle to the housing;
a heating member coupled to the housing and configured to generate heat; and
a herbal receptacle in thermal communication with the heating member, the herbal receptacle having a base and a wall extending from the base to define a receptacle cavity for receiving the herbal products, the herbal receptacle receiving heat from the heating member for vaporizing the herbal products.

2. The vaporizer device of claim 1, wherein the housing is rotatable relative to the bottle between an engaged configuration and a disengaged configuration, the threads of the housing being engaged with the threaded bottle connector when in the engaged configuration, the threads of the housing being disengaged from the threaded bottle connector when in the disengaged configuration.

3. The vaporizer device of claim 1, wherein the heating member includes an elongate heating rod.

4. The vaporizer device of claim 3, further comprising a thermal conduit connected to the herbal receptacle, the thermal conduit defining a thermal opening, the elongate heating rod being insertable into the thermal opening.

5. The vaporizer device of claim 1, further comprising a power unit in electrical communication with the heating member for supplying electrical power to the heating member.

6. The vaporizer device of claim 5, further comprising a temperature regulator in electrical communication with the heating member and the power unit, the temperature regulator being configured to control the flow of electrical power from the power unit to the heating member to maintain the temperature of the herbal receptacle within a vaporizing temperature range.

7. The vaporizer device of claim 6, further comprising a temperature indicator in operative communication with the temperature regulator, the temperature indicator configured to emit a temperature signal when the temperature of the receptacle is in the vaporizing temperature range.

8. The vaporizer device of claim 5, further comprising a power switch in operative communication with the power unit, the power switch being disposable in an ON position, and an OFF position.

9. The vaporizer device of claim 8, further comprising a power indicator in operative communication with the power switch, the power indicator being configured to emit a power signal when the power switch is in the ON position.

10. The vaporizer device of claim 1, wherein the wall is cylindrical.

11. The vaporizer device of claim 1, wherein the housing defines a housing cavity, the heating element being disposed within the housing cavity.

12. The vaporizer device of claim 1, wherein the herbal receptacle is formed from a metallic material.

13. The vaporizer device of claim 1, wherein the housing is formed from a plastic material.

14. A vaporizer device for vaporizing herbal products for inhalation by a user through a delivery apparatus having a delivery connector, the vaporizer device comprising:
a housing having a housing connector configured to be engageable with the delivery connector to connect the delivery apparatus to the housing;
a herbal receptacle coupled to the housing, the herbal receptacle having a base and a wall extending generally orthogonally from the base to define a receptacle cavity for receiving the herbal products; and
a heating member in thermal communication with the herbal receptacle, the heating member configured to heat the herbal receptacle for vaporizing the herbal products therein.

15. The vaporizer device of claim 14, wherein the heating member includes an elongate heating rod.

16. The vaporizer device of claim 15, further comprising a thermal conduit connected to the herbal receptacle, the thermal conduit defining a thermal opening, the elongate heating rod being insertable into the thermal opening.

17. The vaporizer device of claim 14, further comprising a power unit in electrical communication with the heating member for supplying electrical power to the heating member.

18. The vaporizer device of claim 17, further comprising a temperature regulator in electrical communication with the heating member and the power unit, the temperature regulator being configured to control the flow of electrical power from the power unit to the heating member to maintain the temperature of the herbal receptacle within a vaporizing temperature range.

19. The vaporizer device of claim 17, further comprising a power switch in operative communication with the power unit, the power switch being disposable in an ON position, and an OFF position.

20. A method of vaporizing an herbal product, the method comprising the steps of:
a) providing a collection bottle and a vaporizing device, the collection bottle including a bottle wall defining a collection enclosure and a threaded bottle connector, the vaporizing device including:
a housing having a connector portion defining threads configured to be engageable with the threaded bottle connector to connect the bottle to the medical receptacle;
a herbal receptacle connected to the housing, the herbal receptacle having a base and a wall extending from the base to define a receptacle cavity for receiving the herbal products; and
a heating member in thermal communication with the herbal receptacle, the heating member configured to heat the herbal receptacle for vaporizing the herbal products therein;
b) puncturing an inhaling aperture within the bottle wall adjacent an end portions of the collection bottle; and
c) connecting the collection bottle to the vaporizing device by engaging the threaded bottle connector to the connector portion of the housing.
